(19) United States
(12) Patent Application Publication
(10) Pub. No.: US 2012/0088186 A1
(43) Pub. Date: Apr. 12, 2012

(54) Catalyst and Method for the Electrochemical Oxidation of Methane

(75) Inventor: Yu-Wei Lu, Orsay (FR)

(73) Assignees: UNIVERSITE PARIS SUD XI, Orsay (FR); CENTRE NATIONAL DE LA RECHERCHE SCIENTIFIQUE, Paris (FR)

(21) Appl. No.: 13/318,037

(22) PCT Filed: Apr. 23, 2010
(86) PCT No.: PCT/FR2010/000328

§ 371 Date: Dec. 6, 2011

(30) Foreign Application Priority Data
Apr. 28, 2009 (FR) 09 02054

(51) Int. Cl. H01M 4/92; B05D 5/12; H01M 4/64; C07C 29/48; B01J 27/24; B01J 27/188; B01J 27/132; B01J 21/06; C01B 31/20; B01J 31/34

(52) U.S. Cl. 429/521; 423/437.1; 427/115; 429/524; 502/167; 502/171; 502/200; 502/210; 502/228; 502/254; 568/910

(57) ABSTRACT

The invention relates to a catalyst, to the use thereof for the electrochemical conversion of methane to methanol and for the direct electrochemical conversion of methane to CO₂. The invention also relates to an electrode, in particular for a fuel cell including such a catalyst, as well as to a method for manufacturing such an electrode. The invention further relates to a fuel cell including said catalyst or said electrode. The catalyst according to the invention includes a platinum precursor (II), and optionally a metal-ion precursor M supported by particles of a heteropolyanion (HPA). The invention can be used in particular in the field of the electrochemical oxidation of methane into methanol or CO₂.

The invention relates to a catalyst and to its use in the electrochemical conversion of methane to methanol and in the direct electrochemical conversion of methane to CO₂. It also relates to an electrode, in particular a fuel cell electrode, comprising such a catalyst and to a process for the manufacture of such an electrode. It also relates to a fuel cell comprising this catalyst or this electrode.

Fuel cells make possible the conversion of the chemical energy of a fuel into electrical energy. The most well known of the fuel cells is the hydrogen cell, that is to say in which the fuel is hydrogen. Such a cell uses a proton-exchange membrane as electrolyte, which restricts the operating temperature to less than 90° C. It is currently the only fuel cell intended for an onboard application, that is to say an application where it can be transported, as for motor vehicles, residential or tertiary production and cogeneration, miniature hydrogen cells for portable telephones, portable computers, camcorders, and the like.

Furthermore, as energy source, methane is used as fuel for motor vehicles.

The use of natural gas produces CO₂, which is a greenhouse gas.

On the other hand, the use of methane originating from the natural fermentation of plants does not increase the amount of CO₂ in the air and is thus less damaging to the environment.

Methane originating from the natural fermentation of plant waste constitutes a source of energy which is renewable, clean and abundant. It is the only natural fuel which is easy to obtain without any transformation before it is used. This quality renders the use thereof advantageous, much more than that of hydrogen and methanol.

Methane is a completely synthetic molecule and the oxidation thereof is difficult at low temperature.

Such an oxidation requires the use of a catalyst.

However, to date, no known catalyst allows the direct conversion of methane to CO₂ or even the conversion of methane to methanol by an electrochemical process.

Heteropolyanions of the Keggin family (for example: H₄SiW₁₂O₄₀) and of the Dawson family (for example: K₆P₂W₁₈O₆₂) which are used as catalysts for electrochemical reduction of H⁺ to H₂ and of oxygen to H₂O, are also known. These heteropolyanions are also known to be catalysts for the electrochemical reduction of nitrates and nitrites to nitrogen.

However, these catalysts do not make it possible to oxidize methane to methanol or even to CO₂.

In the same way, compounds of platinum with an oxidation state of II are known for the reduction of H³⁰ to H₂ or the oxidation of H₂ to H⁺ by the electrochemical route; they are very quickly converted to platinum with an oxidation state of zero. In all cases, these catalysts are inactive for the oxidation of methane to methanol or of methane to CO₂.

Other catalysts, such as ruthenium, in particular complexed by ligands, are known for reactions for the hydrogenation of organic molecules. Ni compounds with an oxidation state of II and cobalt compounds with an oxidation state of II are known for the use thereof for the reduction of H⁺ to H₂ and iron with an oxidation state of II is also known as catalyst for the reduction of molecular oxygen but they are ineffective for the conversion of methane to methanol or of methane to CO₂.

Compounds of the Ag ion with an oxidation state of I are not regarded as catalysts.

The invention aims at providing a catalyst which makes possible the oxidation of methane to methanol or the direct oxidation of methane to CO₂, which makes it possible, in the case of the conversion of methane to CO₂, to use this catalyst in particular for fuel cells for onboard applications, that is to say applications in motor vehicles, portable telephones, electric generating sets, portable computers, and the like, as replacement for hydrogen fuel cells.

To this end, the invention provides a catalyst, characterized in that it comprises a platinum(II) precursor and optionally a precursor of metal ion(s) M which is (are) supported on particles of a hetero-polyanion HPA, and in that the platinum(II) precursor is a platinum precursor having an oxidation state of II which is optionally complexed by an organic or inorganic ligand,
M is a metal ion chosen from Ag⁺, Ru²⁺, Ni²⁺, Co²⁺, Fe²⁺ and the mixtures of two or more of these,
the heteropolyanion HPA is chosen from H₄SiW₁₂O₄₀ and K₆P₂W₁₈O₆₂.

These heteropolyanions are the most effective of the heteropolyanions among H₄SiW₁₂O₄₀, K₃PW₁₂O₄₀, K₃PMo₁₂O₄₀, K₆P₂W₁₈O₆₂ and K₆P₂W₁₂Mo₆O₆₂.

In a first embodiment, the catalyst of the invention does not comprise metal ion(s) M.

In a second embodiment, the catalyst of the invention comprises a metal ion M or metal ions M.

In all the embodiments of the catalyst of the invention, the platinum(II) precursor is chosen from platinum chloride of formula PtCl₂, bipyridinedichloroplatinum ((Bipy)PtCl₂) of following formula:

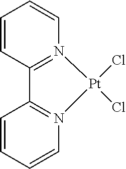

and tetraammineplatinum chloride of formula Pt(NH₃)₄Cl₂.

Also in all the embodiments of the catalyst of the invention, the platinum(II) precursor is preferably (Bipy)PtCl₂ or Pt(NH₃)₄Cl₂.

In the second embodiment of the catalyst of the invention, the precursor of metal ion(s) is preferably a precursor of the Ag⁺ ion.

Still in all the embodiments of the invention, the preferred heteropolyanion HPA is K₆P₂W₁₈O₆₂ and H₄SiW₁₂O₄₀.

The invention also provides for the use of the catalyst of the invention according to these two embodiments for the conversion of methane to methanol.

The invention also provides for the use of the catalyst of the invention according to the second embodiment for the direct conversion of methane to CO₂.

The invention also provides for an electrode, in particular for a fuel cell, characterized in that it comprises a support made of a material which conducts electrons, on at least one surface of which is deposited a catalyst of the invention according to the second embodiment.

Preferably, the material which conducts electrons is chosen from bulk glassy carbon, a carbon fabric, a carbon felt or a sponge of titanium metal.

The invention also provides a process for the manufacture of an electrode according to the invention, characterized in that it comprises the following steps:

(a) dissolution of a heteropolyanion HPA chosen from H₄SiW₁₂O₄₀ and K₆P₂W₁₈O₆₂ in a solvent chosen from a linear or branched C₁ to C₄ alcohol, a mixture of linear or branched C₁ to C₄ alcohols or a mixture of water and of at least one linear or branched C₁ to C₄ alcohol,

(b) deposition of the solution obtained in step (a) on at least one surface of a support made of a material which conducts electrons,

(c) evaporation of the solvent from the solution deposited in step (b),

(d) spraying, over the surface covered with hetero-polyanion obtained in step (c), the solution comprising a platinum precursor with an oxidation state of II, optionally complexed by an organic or inorganic ligand, and a precursor of metal ion(s) M chosen from Ag⁺, Ru²⁺, Co²⁺, Ni²⁺, Fe²⁺ and the mixtures of two or more of these, in a solvent chosen from water, a mixture of water and of at least one linear or branched C₁ to C₄ alcohol and a mixture of linear or branched C₁ to C₄ alcohols,

(e) evaporation of the solvent from the solution sprayed in step (d).

Preferably, the solvent in step (a) and step (c) is isopropanol.

Preferably again, the material which conducts electrons is chosen from bulk glassy carbon, a carbon felt or fabric and a sponge of titanium metal.

Preferably again, the platinum precursor with an oxidation state of II is chosen from PtCl₂, (Bipy)PtCl₂ and Pt(NH₃)₄Cl₂.

More preferably, the platinum precursor with an oxidation state of II is chosen from (Bipy)PtCl₂ and Pt(NH₃)₄Cl₂.

Preferably again, the precursor of metal ion(s) M is a precursor of Ag⁺ ion(s).

Still preferably, the heteropolyanion is K₆P₂W₁₈O₆₂ or H₄SiW₁₂O₄₀.

Preferably again, the material which conducts electrons is a sponge of titanium metal or a carbon felt or fabric.

The invention also provides a process for the transformation of methane to methanol, characterized in that it comprises a step of use of a catalyst of the invention according to the first embodiment.

The invention also provides a process for the direct oxidation of methane to CO₂, characterized in that it comprises a step in which the methane CH₄ is brought into contact with a catalyst of the invention according to the second embodiment.

Finally, the invention provides a fuel cell, characterized in that it comprises a catalyst of the invention according to the second embodiment or an electrode according to the invention or an electrode obtained by the process according to the invention.

A better understanding of the invention will be obtained and other characteristics and advantages of the invention will become more clearly apparent on reading the explanatory description which follows.

The catalyst according to the invention is a catalyst composed of a support, itself composed of particles of a heteropolyanion, denoted HPA, of the Keggin or Dawson family, on which are deposited particles of a platinum precursor with an oxidation state of II and optionally a precursor of metal ion(s) M.

The Keggin heteropolyanion has the formula H₄SiW₁₂O₄₀ and the Dawson heteropolyanion has the formula K₆P₂W₁₈O₆₂.

Thus, in a first embodiment, the catalyst of the invention is composed of the Keggin or Dawson heteropolyanion support, on which is deposited platinum with an oxidation state of II, also denoted Pt(II) or platinum(II). These notations represent as well a precursor in which the platinum has an oxidation state of II in the salt form as a precursor in which the platinum is complexed by one or more ligands.

In the invention, it is preferable to use, as platinum precursor with an oxidation state of II, platinum chloride of formula PtCl₂ or tetraammineplatinum chloride of formula Pt(NH₃)₄Cl₂ or a platinum complex which is (Bipy)PtCl₂ of following formula:

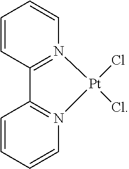

With the catalyst of the invention, the methane becomes fixed by bonding to the platinum which is deposited on the heteropolyanion, to form methanol by the electrochemical route.

In a preferred embodiment, the catalyst of the invention additionally comprises a precursor of metal ion(s) denoted M. This (these) metal ion(s) is (are) preferably chosen from Ag⁺, Ru²⁺, Co²⁺, Ni²⁺ and Fe²⁺ ion(s) and the mixtures of two or more of these.

It is very particularly preferable to use Ag⁺ ions to form the catalyst of the invention.

The number of platinum(II) ions and metal ion(s) M depends on the heteropolyanion and on the valency of the metal ion. Thus, when the metal ion is Ag⁺ and when the heteropolyanion HPA is the heteropolyanion of the Keggin family, the number of Ag⁺ ions deposited is twice the number of Pt²⁺ ions deposited.

On the other hand, when the metal ion is Ru²⁺ or Co²⁺ or Ni²⁺ or Fe²⁺, the number of these metal ions deposited is equal to the number of Pt²⁺ ions deposited.

When the heteropolyanion is a Dawson heteropolyanion and when the metal ion is Ag⁺, the number of Ag⁺ ions deposited on the heteropolyanion is four Ag⁺ ions per one Pt²⁺ ion.

When the metal ion is Ru²⁺ or Co²⁺ or Ni²⁺ or Fe²⁺, the number of metal ions deposited on the heteropolyanion is two metal ions per one Pt²⁺ ion.

With this second embodiment, which is a preferred embodiment, of the catalyst of the invention, either the transformation of methane to methanol or the direct conversion of methane to CO₂ is obtained. In this direct conversion reaction, the methane becomes fixed by bonding to the platinum to give the methanol, the methanol being subsequently oxidized by the metal ions M to give CO₂.

Preferably, in this reaction for the direct conversion of methane to CO₂, use is made of Pt²⁺ metal ions and of metal ions as defined above and, as support, of a Dawson heteropolyanion, which has a greater reactivity than the Keggin heteropolyanion. However, the Dawson heteropolyanion is insoluble in an alcohol, which makes it more difficult to employ in a fuel cell. This is why, in some cases, it may be preferable to use a Keggin heteropolyanion, which has a lower reactivity but which is easier to dissolve as it is soluble in an alcohol.

The catalyst of the invention makes it possible to use very little platinum, which is also one of its advantages.

This is because deposited amounts of two micrograms per square centimeter of surface area of the hetero-polyanion are sufficient to obtain the catalyst of the invention, according to the first or according to the second embodiment.

The amount of platinum used is one Pt(II) ion per 50 to 100 HPA ions. The more platinum there is, the faster the oxidation reaction. If this ratio is less than 10, partial oxidation of the CH₄ is favored.

With regard to the amount of metal ions deposited, it can vary according to the oxidation state of these ions. The metal ion serves to preserve the layer of the heteropolyanion. It is exchanged with the counterions of HPA until the charges carried by the HPA have been completely neutralized.

Thus, the catalyst according to the second embodiment of the invention is of particular use in the manufacture of an electrode, in particular of a fuel cell. This electrode is composed of a support made of a material which conducts electrons, on at least one surface of which is deposited a catalyst according to the invention.

The preferred material which conducts electrons is chosen from bulk glassy carbon, a carbon fabric, a carbon felt and a sponge made of titanium metal.

Preferably, for use as fuel cell electrode, the preferred support made of material which conducts electrons is a sponge of titanium metal.

However, a carbon felt or a carbon fabric are also preferred.

The electrode according to the invention, in particular for a fuel cell, can be manufactured by a process which comprises the steps of dissolving the heteropolyanion in a linear or branched C₁ to C₄ alcohol or in a mixture of linear or branched C₁ to C₄ alcohols or in a mixture of water and of at least one linear or branched C₁ to C₄ alcohol, of the chosen heteropolyanion.

Use will preferably be made, as solvent, of isopropanol.

The solution obtained is then deposited, for example by spraying, on at least one surface of a support made of material which conducts electrons and the solvent is evaporated; the solvent is advantageously freely evaporated at ambient temperature.

Preferably, the support made of a material which conducts electrons is bulk glassy carbon or fabrics or felts of glassy carbon. Most preferably, the support is a sponge of titanium metal.

A solution composed of platinum precursor with an oxidation state of II, preferably PtCl₂ or (Bipy)PtCl₂ or Pt(NH₃)₄Cl₂, and optionally metal ion(s) M chosen from Ag⁺, Ru²⁺, Ni²⁺, Co²⁺ or Fe²⁺ in a solvent is then sprayed over the surface of the support made of a material which conducts electrons covered with the heteropolyanion HPA. The solvent for the platinum(II) precursor and optionally for the metal ion(s) M is chosen from water or a mixture of water and of at least one linear or branched C₁ to C₄ alcohol, a linear or branched C₁ to C₄ alcohol or a mixture of two or more linear or branched C₁ to C₄ alcohols.

Preferably, this solvent is isopropanol, that is to say the same solvent as that which was used to solubilize the heteropolyanion HPA.

This solvent is subsequently evaporated, advantageously freely in the air at ambient temperature.

Of course, as will be recognized by a person skilled in the art, the surface of the material which conducts electrons must be perfectly clean in order to avoid any contamination of the catalyst or catalysts.

Use will preferably be made, as platinum precursor with an oxidation state of II, of PtCl₂, (Bipy)PtCl₂ or Pt(NH₃)₄Cl₂.

When a metal ion or metal ions is (are) additionally present in the catalyst of the invention, this (these) metal ion(s) is (are) preferably Ag⁺ ions.

With regard to the heteropolyanion HPA, it is also, as for the catalyst of the invention, preferably a heteropolyanion of the Dawson or Keggin family.

The invention also provides a fuel cell which comprises a catalyst according to the invention as described above, or an electrode according to the invention as described above, or an electrode obtained by the process according to the invention as described above.

The catalyst of the invention, or the electrode of the invention or the electrode obtained by the process of the invention, when the catalyst does not comprise metal ion(s) M, is intended in particular for the implementation of a process for the transformation of methane to methanol.

When the catalyst of the invention, or when the electrode of the invention or when the electrode obtained by the process of the invention comprising this catalyst, is a catalyst in which a metal ion M or metal ions M is (are) present, preferably, this catalyst and this electrode are particularly appropriate for the implementation of a process for the direct oxidation of methane to CO₂, in particular when the metal ion(s) is (are) Ag⁺.

The invention also relates to all devices, in particular mobile devices, which would comprise a fuel cell according to the invention or which would use a catalyst or an electrode according to the invention or an electrode obtained by the process according to the invention.

Such devices, by way of examples, are motor vehicles, portable telephones or portable computers or also electric generating sets.

In order to make the invention better understood, a description will now be given of several implementation examples thereof, as purely illustrative and nonlimiting example.

### EXAMPLE 1

### Preparation of an Electrode for the Analytical Study of the Oxidation of Methane

Two μL of a solution of a Keggin heteropolyanion (H₄SiW₁₂O₄₀) in isopropanol at a concentration of 10 mg per mL or of a Dawson heteropolyanion (K₆P₂W₁₈O₆₂) in water/isopropanol at a concentration of 5 mg per mL are deposited on a polished surface of glassy carbon with a diameter of 3 millimeters. After the evaporation of the isopropanol solvent, 2 μL of a mixture comprising a precursor of platinum ions with an oxidation state of II (in this instance PtCl₂, Pt(Bipy)Cl₂ or Pt(NH₃)₄Cl₂) at a concentration of 2×10⁻⁵M and a precursor of metal ions Ag⁺ in the form of AgNO₃ at a concentration of 0.08M are deposited. The solvent used is water or a water/isopropanol mixture. This solvent is subsequently evaporated. Under neon light, an areola colored several colors when the film is thin is seen with H₄SiW₁₂O₄₀ or an offwhite areola is seen with K₆P₂W₁₈O₆₂.

### EXAMPLE 2

### Test of the Electrode Obtained in Example 1 for the Direct Conversion of Methane to CO₂

The electrode obtained in example 1 is dipped into a pH 3 buffer solution of Na₂SO₄/H₂SO₄ and cycled between 0.9 and 1.7 volts, with respect to a saturated calomel electrode (SCE), under argon until the current has stabilized. It takes approximately from 30 minutes to 1 hour. The solution is then saturated by diffusion of methane at the surface. Saturation is slow. Catalysis can last more than 3 hours.

This test is carried out at ambient temperature, that is to say at a temperature of between 15° C. and 25° C.

### EXAMPLE 3

### Preparation of an Electrode of High Surface Area with the Catalyst According to the Invention

A solution of a Keggin heteropolyanion (H₄SiW₁₂O₄₀) in isopropanol at a concentration of 10 mg per mL or of a Dawson heteropolyanion (K₆P₂W₁₈O₆₂) in water/isopropanol at a concentration of 5 mg per mL is sprayed over a 3 cm² polished glassy carbon surface.

The isopropanol is evaporated and then a mixture of the platinum precursor with an oxidation state of II at a concentration of 2×10⁻⁵M and of the precursor of metal ions Ag⁺ at a concentration of 8×10⁻²M is projected over the glassy carbon surface on which the hetero-polyanion had been deposited.

The solvent, which is also water-isopropanol here, is subsequently evaporated.

### EXAMPLE 4

### Test of the Electrode of Example 3 for the Direct Conversion of Methane to CO₂

The electrode obtained in example 3 is immersed in the compartment of a two-compartment cell filled with 0.1M Na₂SO₄ pH 3 buffer solution. The other compartment of the cell comprises an electrode made of bulk platinum. From the contact of the methane with the catalyst, the potential difference between the bulk platinum electrode (positive pole) and the electrode according to the invention (negative pole) increases up to 0.4 volt, which clearly shows the fixing of the CH₄ to the catalyst.

TABLE 1
Potential difference between the positive pole
(Pt) and the negative pole (GC, with or without the
catalyst PtM(HPA))

| | Without | With |
|---|---|---|
| Air | 0.350 V | 0.151 V |
| Ar | 0.432 V | 0.152 V |
| CH₄ | 0.432 V | 0.428 V |

Experimental conditions: 0.1M Na₂SO₄/H₂SO₄ pH 3, SCE, Pt,
M = Ag,
HPA = H₄SiW₁₂O₄₀.

If the two electrodes are connected by a copper conductor and an ammeter in series, a weak current circulates in the cell. The presence of CO₂ in the solution (detected by GC-MS) confirms the direct conversion of methane to CO₂.

Here again, this test is carried out at ambient temperature.

### EXAMPLE 5

### Preparation of an Electrode for the Methane Fuel Cell

1 mL of a solution of a Keggin heteropolyanion (H₄SiW₁₂O₄₀) in isopropanol at a concentration of 10 mg per mL or of a Dawson heteropolyanion (K₆P₂W₁₈O₆₂) in water/isopropanol at a concentration of 5 mg per mL is projected over a 25 cm² titanium sponge. The isopropanol is evaporated. A solution comprising 0.1 mL of a solution comprising platinum ions with an oxidation state of II at a concentration of 10⁻⁴M, added in the form of the precursor PtC1₂, Pt(Bipy)Cl₂ or Pt(NH₃)₄Cl₂, and 0.1 mL of a solution comprising metal ions Ag⁺ at a concentration of 10⁻¹M, in 0.3 mL of isopropanol, is then deposited on the surface of the titanium foam covered with the heteropolyanion.

This electrode is fitted to a hydrogen fuel cell. This cell, although having an outlet for the discharge of the CO₂ produced by the combustion of the methane, after an induction period, is operational. The closed circuit current strength remains stable for more than 5 minutes and then it slowly decreases until halting due to the CO₂ which has accumulated in the cell. The performance cannot be determined under these conditions.

This example shows that the catalyst of the invention and the electrode of the invention can be used in a fuel cell operating with methane at ambient temperature.

Generally, the above examples show that the catalyst of the invention makes possible the conversion of methane to methanol or to CO₂.

Tests carried out at 80° C. show that the catalyst of the invention is stable at this temperature and still makes it possible to convert methane to methanol or to CO₂.

1. A catalyst comprising a platinum(II) precursor and optionally a precursor of metal ion(s) M which is (are) supported on particles of a heteropolyanion HPA, and in that:
the platinum(II) precursor is a platinum precursor having an oxidation state of II which is optionally complexed by an organic or inorganic ligand,
M is a metal ion chosen from Ag⁺, Ru²⁺, Ni²⁺, Co²⁺, Fe²⁺ and the mixtures of two or more of these,
the heteropolyanion HPA is chosen from H₄SiW₁₂O₄₀ and K₆P₂W₁₈O₆₂.

2. The catalyst as claimed in claim 1, wherein the catalyst does not comprise metal ion(s) M.

3. The catalyst as claimed in claim 1, wherein the catalyst comprises at least one metal ion M.

4. The catalyst as claimed in claim 1, wherein the platinum(II) precursor is chosen from platinum chloride of formula PtCl₂, bipyridinedichloroplatinum (Bipy)PtCl₂ of following formula: 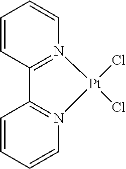
and tetraammineplatinum chloride of formula Pt(NH₃)₄Cl₂.

5. The catalyst as claimed in claim 1, wherein the platinum(II) precursor is (Bipy)PtCl₂ or Pt(NH₃)₄Cl₂.

6. The catalyst as claimed in claim 1, wherein the precursor of metal ion(s) is an Ag⁺ precursor.

7. The catalyst as claimed in claim 1, wherein the heteropolyanion HPA is K₆P₂W₁₈O₆₂.

8. The use of the catalyst as claimed in claim 1 for the conversion of methane to methanol.

9. The use of the catalyst as claimed in claim 1 for the conversion of methane to CO₂.

10. An electrode comprising a support made of a material which conducts electrons, on at least one surface of which is deposited a catalyst as claimed in claim 1.

11. The electrode as claimed in claim 10, wherein the material which conducts the electrons is chosen from bulk glassy carbon, a glassy carbon fabric, a glassy carbon felt or a sponge of titanium metal.

12. A process for the manufacture of the electrode as claimed in claim 10, comprising the following steps:
(a) dissolution of a heteropolyanion HPA chosen from H₄SiW₁₂O₄₀ and K₆P₂W₁₈O₆₂ in a solvent chosen from a linear or branched C₁ to C₄ alcohol, a mixture of linear or branched C₁ to C₄ alcohols or a mixture of water and of at least one linear or branched C₁ to C₄ alcohol,
(b) deposition of the solution obtained in step (a) on at least one surface of a support made of a material which conducts electrons,
(c) evaporation of the solvent from the solution deposited in step (b),
(d) spraying, over the surface coated with heteropolyanion obtained in step (c), the solution comprising a platinum precursor with an oxidation state of II, optionally complexed by an organic or inorganic ligand, and a precursor of metal ion(s) M chosen from Ag⁺, Ru²⁺, Co²⁺, Ni²⁺, Fe²⁺ and the mixtures of two or more of these, in a solvent chosen from water, a mixture of water and of at least one linear or branched C₁ to C₄ alcohol, a linear or branched C₁ to C₄ alcohol and a mixture of linear or branched C₁ to C₄ alcohols, and
(e) evaporation of the solvent from the solution sprayed in step (d).

13. The process as claimed in claim 12, wherein the solvent in step (a) and in step (c) is isopropanol.

14. The process as claimed in claim 12 wherein the material which conducts electrons is chosen from bulk glassy carbon, a carbon felt, a carbon fabric and a sponge of titanium metal.

15. The process as claimed in claim 12 wherein the platinum precursor with an oxidation state of II is chosen from PtCl₂, (Bipy)PtCl₂ and Pt(NH₃)₄Cl₂.

16. The process as claimed in claim 12 wherein the platinum compound with an oxidation state of II is chosen from (Bipy)PtCl₂ and Pt(NH₃)₄Cl₂.

17. The process as claimed in claim 12 wherein the metal ion(s) M of the precursor of metal ion(s) is (are) Ah⁺.

18. The process as claimed in claim 12 wherein the heteropolyanion is K₆P₂W₁₈O₆₂.

19. The process as claimed in claim 12 wherein the material which conducts electrons is a sponge of titanium metal or a carbon felt or fabric.

20. A process for the transformation of methane to methanol, wherein the process comprises a step of use of a catalyst as claimed in claim 1.

21. A process for the direct oxidation of methane to CO₂, wherein the process comprises a step in which CH₄ is brought into contact with a catalyst as claimed in claim 1.

22. A fuel cell, comprising a catalyst as claimed in claim 1.

23. A fuel cell comprising an electrode as claimed in claim 11 or an electrode.

24. A fuel cell obtained by the process as claimed in claim 12.
